(19) United States
(12) Patent Application Publication
(10) Pub. No.: US 2006/0246498 A1
(43) Pub. Date: Nov. 2, 2006

(54) HNRPLL polypeptides, polynucleotides encoding same and compositions and methods of using same

(75) Inventor: Dafna Benayahu, Herzlia (IL)

Correspondence Address: Martin D. Moynihan;PRTSI, Inc., P.O. Box 16446, Arlington, VA, 22215, US

(73) Assignee: Ramot At Tel Aviv University Ltd.

(21) Appl. No.: 11/411,769

(22) Filed: Apr. 27, 2006

Related U.S. Application Data
(60) Provisional application No. 60/675,486, filed on Apr. 28, 2005.

(51) Int. Cl. C12Q 1/68; G01N 33/567; C12P 21/06; C07H 21/04; C07K 14/705; C07K 16/28

(52) U.S. Cl. 435/6; 435/69.1; 435/320.1; 435/325; 530/350; 536/23.5; 530/388.1; 514/12; 435/7.2

(57) ABSTRACT

An isolated polynucleotide comprising a nucleic acid sequence encoding a stromal RNA regulating factor polypeptide being specifically expressed in mesenchymal stem cells and not in skeletal muscle cells is disclosed. Methods of detecting same are disclosed as well as methods of using same for modulating RNA in mesenchymal stem cells.

### RELATED APPLICATION

This Application claims the benefit of U.S. Provisional Patent Application No. 60/675,486 filed on Apr. 28, 2005, the contents of which are hereby incorporated in its entirety.

### FIELD AND BACKGROUND OF THE INVENTION

The present invention relates to novel HNRPLL polypeptides, polynucleotides, compositions comprising same and uses thereof.

Bone marrow contains at least two types of stem cells, hematopoietic stem cells and stem cells of non-hematopoietic tissues. The latter types of cells are generally referred to as mesenchymal stem cells or marrow stromal cells (MSCs). MSCs are of interest because they are capable of differentiating under appropriate conditions into various lineages, including fibroblastic, endothelial, osteogenic and chodrogenic cells and adipocytes. In addition, they are easily isolated from a small aspirate of bone marrow, and they readily generate single-cell derived colonies. Single-cell derived colonies of MSCs can be expanded through as many as 50 population doublings in about 10 weeks.

MSCs are referred to as mesenchymal stem cells because they are capable of differentiating into multiple mesodermal tissues, including bone, cartilage, cardiac and skeletal muscle, glial cells, neurons and hepatocytes suggesting that MSCs may be capable of overcoming germ layer commitment.

As a result of their unique features, it has been suggested that these cells may hold the promise of changing the face of cell transplantation, by replacing or restoring tissue that has been damaged by disease or injury. Thus, diseases that might be treated by transplanting human ES-derived cells include Parkinson's disease, diabetes, traumatic spinal cord injury, Purkinje cell degeneration, Duchenne's muscular dystrophy, heart failure, and osteogenesis imperfecta.

Stem cell differentiation depends on the coordinated regulatory mechanisms that affect gene expression including both RNA transcription and mRNAs processing. Heterogeneous ribonucleoproteins (hnRNPs) control RNA processing from the nascent transcripts emerging from RNA polymerase II transcription unit, until mature mRNAs are formed. hnRNP proteins are among the most abundant proteins in the nucleus. They have a modular structure containing one or more RNA-binding domain and at least one other domain that is important for interaction with other proteins. The association of pre-mRNAs with hnRNP proteins prevents the formation of short secondary structures dependent on base pairing of complementary regions, thereby making pre-mRNAs accessible for interaction with other macromolecules. Thus, pre-mRNAs associated with hnRNP proteins present a more uniform substrate for further processing steps than would free, unbound pre-mRNAs.

hnRNPs function at several levels within cells including alternative mRNA splicing and stabilization of tissue specific mRNAs. They participate in 5′-cap formation, methylation, 3′-end cleavage and poly-adenylation, splicing of introns and probably transportation of mRNA. Analysis of splicing of pre-mRNA from complex transcription units has shown that differences in the relative concentrations of hnRNP proteins can influence the selection of alternatively spliced sites and may contribute to cell type-specific splicing (Weighardt et al, 1996, Bioessays 18(9), 747-756). The significance of protein-RNA interactions is reflected in the findings that several human and other vertebrate genetic disorders are caused by aberrant expression of RNA-binding proteins.

hnRNPs generally comprise at least one RNA-binding motif (RRM). A subfamily of RRM-type RNA binding proteins described initially by Dreyfuss and colleagues (Dreyfuss et al, 1993, Annu. Rev. Biochem. 62, 289-321) includes both hnRNP-L and hnRNP-I. Proteins in this subfamily were demonstrated to regulate tissue specific RNA expression by repressing particular splicing events. These proteins along with ROD1 and matrin contain RRM motifs highly homologous with each other but weakly homologous to the canonical RRM consensus sequence (Polydorides et al, 2000, Proc Natl Acad Sci USA 97 (12), 6350-6355). Identified restricted expression of ROD1 protein and various alternatively spliced forms of hnRNP-I were suggested to affect tissue specific expression and alternative splicing patterns of RNA (Wollerton et al, 2001, RNA 7:819-832; Yamamoto et al, 1999, Molec Cell Biology, 19, 3829-3841). It has been demonstrated that ROD1 protein is expressed in hematopoetic cells and participates in the control of their differentiation (Yamamoto et al, 1999, Molec Cell Biology, 19, 3829-3841). A brain-specific isoform of hnRNP-I was discovered in an attempt to characterize RNA binding proteins that mediate cell and tissue specific splicing in neurons (Polydorides et al, 2000, Proc Natl Acad Sci USA 97 (12), 6350-6355; Rahman et al, 2002, Genomics. 80(3), 245-9). HnRNP-I was also demonstrated to regulate gene expression in specific tissues during embryonic development (Davis et al, 2002, Mech Dev. 111(1-2), 143-7). Distinct structure of hnRNP-I splice forms impacts upon their expression in a tissue specific manner and provides their stronger or weaker repressive activity accordingly.

Although, tissue specific hnRNPs have been isolated and postulated as playing a role in the regulation of differentiation, none have been identified in bone marrow stromal cells.

### SUMMARY OF THE INVENTION

According to the present invention there is provided an isolated polynucleotide comprising a nucleic acid sequence encoding an HNRPLL polypeptide, the polypeptide being specifically expressed in mesenchymal stem cells and not in skeletal muscle cells.

According to another aspect of the present invention there is provided an isolated polynucleotide as set forth in SEQ ID NO: 1.

According to yet another aspect of the present invention there is provided an isolated polynucleotide as set forth in SEQ ID NO: 3.

According to still another aspect of the present invention there is provided an oligonucleotide capable of specifically hybridizing to an isolated polynucleotide as set forth in SEQ ID NO: 1 and not to an isolated polynucleotide as set forth in SEQ ID NO: 3.

According to an additional aspect of the present invention there is provided an oligonucleotide capable of specifically hybridizing to the isolated polynucleotide as set forth in SEQ ID NO: 3 and not to the isolated polynucleotide as set forth in SEQ ID NO: 1.

According to yet an additional aspect of the present invention there is provided an isolated polypeptide comprising an amino acid sequence encoding an HNRPLL polypeptide, the polypeptide being specifically expressed in mesenchymal stem cells and not in skeletal muscle cells.

According to still an additional aspect of the present invention there is provided an isolated polypeptide as set forth in SEQ ID NO: 2.

According to a further aspect of the present invention there is provided an isolated polypeptide as set forth in SEQ ID NO: 4.

According to yet a further aspect of the present invention there is provided an antibody capable of specifically recognizing an isolated polypeptide as set forth in SEQ ID NO: 2 and not an isolated polypeptide as set forth in SEQ ID NO: 4.

According to still a further aspect of the present invention there is provided an antibody capable of specifically recognizing an isolated polypeptide of as set forth in SEQ ID NO: 4 and not an isolated polypeptide as set forth in SEQ ID NO: 2.

According to still a further aspect of the present invention there is provided a method of modulating RNA processing in mesenchymal stem cells, comprising contacting the mesenchymal stem cells with an agent capable of regulating an expression and/or activity of the isolated polypeptide comprising an amino acid sequence encoding an HNRPLL polypeptide, the polypeptide being specifically expressed in mesenchymal stem cells and not in skeletal muscle cells, thereby regulating RNA processing in mesenchymal stem cells.

According to still a further aspect of the present invention there is provided a method of identifying a mesenchymal stem cell comprising identifying in a biological sample a cell expressing the polynucleotide comprising a nucleic acid sequence encoding an HNRPLL polypeptide, the polypeptide being specifically expressed in mesenchymal stem cells and not in skeletal muscle cells, the cell being the mesenchymal stem cell.

According to still a further aspect of the present invention there is provided a method of identifying a mesenchymal stem cell comprising identifying in a biological sample a cell expressing the polypeptide comprising an amino acid sequence encoding an HNRPLL polypeptide, said polypeptide being specifically expressed in mesenchymal stem cells and not in skeletal muscle cells, the cell being the mesenchymal stem cell.

According to further features in preferred embodiments of the invention described below, the nucleic acid sequence does not comprise SEQ ID NO:14.

According to still further features in the described preferred embodiments the nucleic acid sequence does not comprise SEQ ID NO:15.

According to still further features in the described preferred embodiments the nucleic acid sequence does not comprise SEQ ID NO:16.

According to still further features in the described preferred embodiments the nucleic acid sequence comprises a nucleic acid segment as set forth in SEQ ID NO:17 at the 3′ terminus of the isolated polynucleotide.

According to still further features in the described preferred embodiments the nucleic acid sequence comprises a nucleic acid segment as set forth in SEQ ID NO:18.

According to still further features in the described preferred embodiments the nucleic acid sequence is as set forth in SEQ ID NO: 1.

According to still further features in the described preferred embodiments the nucleic acid sequence is as set forth in SEQ ID NO: 1.

According to still further features in the described preferred embodiments the nucleic acid sequence is as set forth in SEQ ID NO: 3.

According to still further features in the described preferred embodiments the nucleic acid sequence is as set forth in SEQ ID NO: 1 or SEQ ID NO: 3.

According to still further features in the described preferred embodiments the amino acid sequence is as set forth in SEQ ID NO: 2 or SEQ ID NO: 4.

According to still further features in the described preferred embodiments the regulating is down-regulating.

According to still further features in the described preferred embodiments the agent is selected from the group consisting of an antibody, an antisense, an siRNA, a ribozyme and a DNAzyme.

According to still further features in the described preferred embodiments the agent comprises the oligonucleotide capable of specifically hybridizing to an isolated polynucleotide as set forth in SEQ ID NO: 1 and not to an isolated polynucleotide as set forth in SEQ ID NO: 3.

According to still further features in the described preferred embodiments the agent comprises the oligonucleotide capable of specifically hybridizing to the isolated polynucleotide as set forth in SEQ ID NO: 3 and not to the isolated polynucleotide as set forth in SEQ ID NO: 1.

According to still further features in the described preferred embodiments the antibody is the antibody capable of specifically recognizing an isolated polypeptide as set forth in SEQ ID NO: 2 and not an isolated polypeptide as set forth in SEQ ID NO: 4.

According to still further features in the described preferred embodiments the antibody is the antibody capable of specifically recognizing an isolated polypeptide of as set forth in SEQ ID NO: 4 and not an isolated polypeptide as set forth in SEQ ID NO: 2.

According to still further features in the described preferred embodiments the regulating is up-regulating.

According to still further features in the described preferred embodiments the agent is the isolated polynucleotide comprising a nucleic acid sequence encoding an HNRPLL polypeptide, said polypeptide being specifically expressed in mesenchymal stem cells and not in skeletal muscle cells.

According to still further features in the described preferred embodiments the identifying is effected via the oligonucleotide capable of specifically hybridizing to an isolated polynucleotide as set forth in SEQ ID NO: 1 and not to an isolated polynucleotide as set forth in SEQ ID NO: 3.

According to still further features in the described preferred embodiments the identifying is effected via the oligonucleotide capable of specifically hybridizing to the isolated polynucleotide as set forth in SEQ ID NO: 3 and not to the isolated polynucleotide as set forth in SEQ ID NO: 1.

According to still further features in the described preferred embodiments the identifying is effected via the antibody capable of specifically recognizing an isolated polypeptide as set forth in SEQ ID NO: 2 and not an isolated polypeptide as set forth in SEQ ID NO: 4.

According to still further features in the described preferred embodiments the identifying is effected via the antibody capable of specifically recognizing an isolated polypeptide of as set forth in SEQ ID NO: 4 and not an isolated polypeptide as set forth in SEQ ID NO: 2.

The present invention successfully addresses the shortcomings of the presently known configurations by providing polynucleotides and polypeptides encoding novel isoforms of HNRPLL.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B are autoradiograph images pertaining to sequence analysis and expression of Transcript A (SEQ ID NO: 1) and Transcript B (SEQ ID NO: 3). FIG. 1A depicts a Southern Blot demonstrating the sequence homology of Transcript A, lane 1 and Transcript B, lane 2. Transcripts A and B were initially subjected to restriction analysis and then probed with a cDNA probe cloned by MMS-85. Transcript A generated 3 fragments and transcript B generated 2 fragments. FIG. 1B depicts a Northern blot of total RNA generated from bone marrow stromal cells (MSCs). The RNA was probed with a cDNA cloned by MMS-85. The autoradiograph demonstrates the presence of two transcripts. For both blots, probes were radio-labeled with [³²P] αCTP and exposed to X-ray.

FIG. 2 is a sequence alignment scheme showing the homology between the two cDNA transcripts A and B analyzed with BLAST2 program. The red marked area indicates complete sequence alignment. The blue marked area indicates partial sequence alignement.

FIGS. 3A-E are schematic illustrations of the HNRPLL gene and its alternate transcripts. FIG. 3A depicts the complete gene structure including 14 exons and the alternatively spliced exons. FIG. 3B depicts the pattern of alternative splicing at the first and last exon for the Transcript A (SEQ ID NO: 1); FIG. 3C depicts the pattern of alternative splicing at the first and last exon for Transcript B (SEQ ID NO: 3); FIG. 3D depicts the pattern of alternative splicing at the first and last exon for transcript BC017480 (SEQ ID NO: 13); and FIG. 3E depicts the pattern of alternative splicing at the first and last exon for transcript AK000155 (SEQ ID NO: 11). The data are complementary to Table 3. Starting methionine is marked by asterisk; stop codon is marked by triangle. Exons 1, 5, 7 and 14 are marked by different background patterns representing the areas where the alternative spicing occurs.

FIGS. 4A-C depict exon composition of stromal RNA regulating factor in MSC cells and universal cells. FIG. 4A is a schematic view of exon composition and length in bp (axis Y) of the full length gene. FIG. 4B is an autoradiograph image depicting PCR amplification of the region spanning exons 3-8 using primers SRRF1 (SEQ ID NO: 7) and SRRF2 (SEQ ID NO: 8); FIG. 4C is an autoradiograph image depicting PCR amplification of exon 14 using primers SRRF3 (SEQ ID NO: 9) and SRRF4 (SEQ ID NO: 10); For FIGS. 4B-C, MSC refers to cDNA generated from MSC cells; U refers to cDNA generated from a universal library. The data are complementary to Table 4.

FIG. 5 is a sequence alignment scheme depicting the homology at the protein level for transcript-A (SEQ ID NO: 2) and transcript-B (SEQ ID NO: 4). Protein motifs are marked by different colors (RRM domain, Coiled coil, DNA binding domain [ps00043], WW, PTB, PDZ, SH2 and 14-3-3 protein potential ligand motifs).

FIG. 6 is sequence alignment scheme depicting multiple protein homolgies between transcript-A (SEQ ID NO: 2) and transcript-B (SEQ ID NO: 4) with hnRNP L, hnRNP I and ROD1 using Multalin program. The boxes mark RRM domain.

### DESCRIPTION OF THE PREFERRED EMBODIMENTS

The present invention is of HNRPLL polypeptides, polynucleotides, compositions comprising same and uses thereof. The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Stem cell differentiation depends on the coordinated regulatory mechanisms that affect gene expression including both RNA transcription and mRNAs processing. Heterogeneous ribonucleoproteins (hnRNPs) control RNA processing from the nascent transcripts emerging from RNA polymerase II transcription unit, until mature mRNAs are formed.

hnRNPs function at several levels within cells including alternative mRNA splicing and stabilization of tissue specific mRNAs. They participate in 5′-cap formation, methylation, 3′-end cleavage and poly-adenylation, splicing of introns and probably transportation of mRNA. Analysis of splicing of pre-mRNA from complex transcription units has shown that differences in the relative concentrations of hnRNP proteins can influence the selection of alternatively spliced sites and may contribute to cell type-specific splicing.

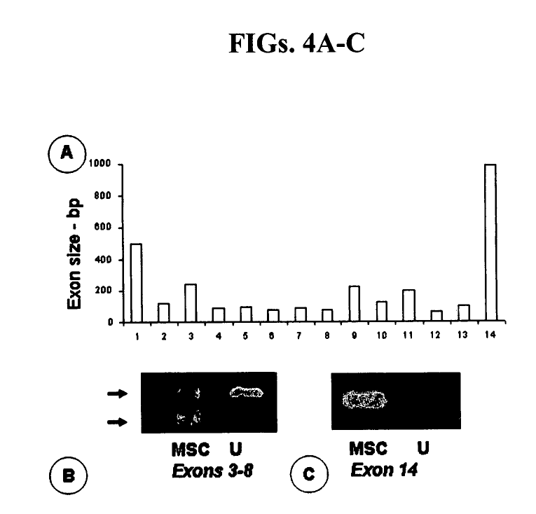

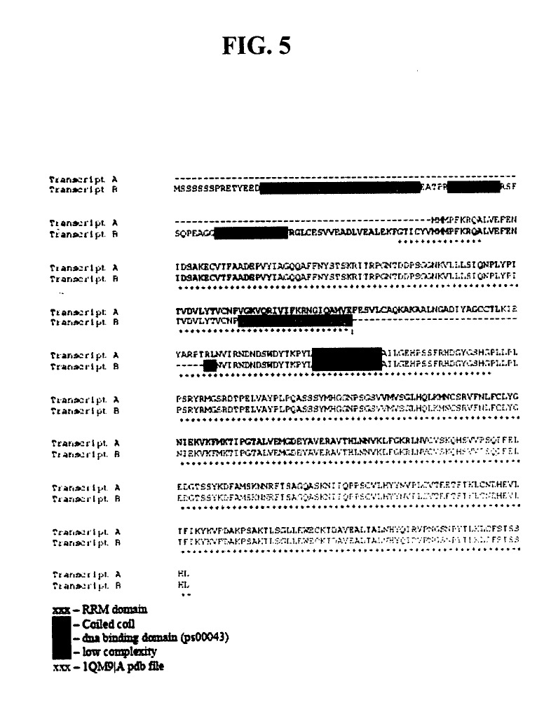

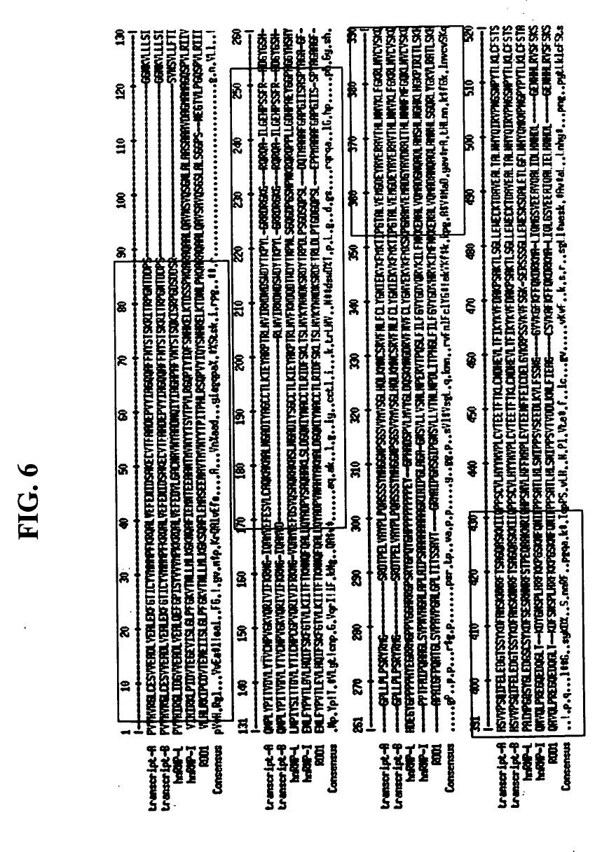

While reducing the present invention to practice the present inventor has uncovered novel alternatively spliced isoforms of HNRPLL (heterogeneous nuclear ribonucleoprotein L-like) which are specifically expressed in mesenchymal stem cells. Such isoforms have been named stromal RNA regulating factors (SRRFs), since as opposed to previously cloned HNRPLLs they show an MSC specific pattern of expression. This suggests that SRRFs may be used as MSC markers (FIGS. 4B-C). Using bioinformatic techniques, the present inventor has shown that these novel isoforms comprise RNA and protein binding motifs (FIG. 5). Furthermore, the present inventor has revealed a high homology between the novel isoforms of the present invention and other hnRNP proteins (FIG. 6). Thus, the isoforms of the present invention may be used for regulating differentiation in such cells along a particular pathway.

Thus, according to one aspect of the present invention, there is provided an isolated polynucleotide comprising a nucleic acid sequence encoding an HNRPLL polypeptide, the polypeptide being specifically expressed in mesenchymal stem cells and not in skeletal muscle cells. The novel isoforms are referred to herein as stromal RNA regulating factors.

As used herein the phrase “HNRPLL polypeptide” refers to a polypeptide expression product of heterogeneous nuclear ribonucleoprotein L-like (HNRPLL) gene (Genebank Accession No. NC_{—}000002: c38683528-38644678).

As mentioned the isolated polynucleotide of this aspect of the present invention is expressed in mesenchymal stem cells and not in other cells such as skeletal muscle cells.

As used herein, the phrase “mesenchymal stem cells” refers to bone marrow, non-hematopoietic pluripotent stem cells, also referred to as stromal cells. The cells may be either isolated or non-isolated and situated either in vivo or ex vivo.

The phrase “skeletal muscle cells” as used herein refers to any cells derived from skeletal muscle. Methods of ascertaining whether the polypeptides of the present invention are specifically expressed in mesenchymal stem cells and not in other cells, such as skeletal muscle cells, nervous system cells, heart cells, kidney cells, liver cells, lung cells and pancreatic cell as described hereinbelow.

It will be appreciated that prior art isoforms of HNRPLL are not specifically expressed in MSCs (i.e. they are also expressed in other cells such as skeletal muscle cells) whilst the isoforms of the present invention are specifically expressed in MSCs.

As used herein the phrase “an isolated polynucleotide” refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

The polynucleotides of the present invention are typically splice variants of the HNRPLL gene.

The phrase “splice variant” refers to alternative forms of RNA transcribed from an HNRPLL gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a polypeptide encoded by a splice variant of an mRNA transcribed from a gene.

The polynucleotides of the present invention may also be allelic variants of the sequence as set forth in GenBank Accession No. NC_{—}000002: c38683528-38644678.

The phrase “allelic variant” refers to two or more alternative forms of a HNRPLL gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

According to an embodiment of this aspect of the present invention, the nucleic acid sequence of the above-described isolated polynucleotide of the present invention does not comprise exon 7 the HNRPLL gene (SEQ ID NO:14).

According to another embodiment of this aspect of the present invention, the nucleic acid sequence of the above-described isolated polynucleotide of the present invention does not comprise a nucleic acid segment of exon 1 of HNRPLL gene (SEQ ID NO:15).

According to yet another embodiment of this aspect of the present invention, the nucleic acid sequence of the above-described isolated polynucleotide of the present invention comprises a nucleic acid segment of exon 14 of HNRPLL gene (SEQ ID NO:17) at the 3′ terminus of the isolated polynucleotide.

Thus, an example of the isolated polynucleotide of the present invention is set forth in SEQ ID NO: 1, which is also referred to herein as Transcript A.

According to another embodiment of this aspect of the present invention, the nucleic acid sequence of the above-described isolated polynucleotide of the present invention does not comprise exon 5 (SEQ ID NO:16).

According to still another embodiment of this aspect of the present invention, the nucleic acid sequence of the above-described isolated polynucleotide of the present invention comprises a nucleic acid segment as set forth in SEQ ID NO:18.

Thus, an example of the isolated polynucleotide of the present invention is set forth in SEQ ID NO: 3, which is also referred to herein as Transcript B.

It will be appreciated that homologues of the sequences described hereinabove are also envisaged by the present invention. Accordingly, the polynucleotides of this aspect of the present invention may have a nucleic acid sequence at least 50%, at least 55%, at least 60%, at least 65%, at least 70%, at least 75%, at least 80%, at least 85%, at least 87%, at least 89%, at least 90% at least 91%, at least 93%, at least 95% or more say 100% identical to SEQ ID NO: 1 or 3, as determined using BlastN software of the National Center of Biotechnology Information (NCBI) using default parameters.

Thus, the present invention encompasses nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Since the polynucleotide sequences of the present invention encode previously unidentified polypeptides, the present invention also encompasses novel polypeptides or portions thereof, which are encoded by the isolated polynucleotides and respective nucleic acid fragments thereof described hereinabove.

Thus, the present invention also encompasses polypeptides encoded by the polynucleotide sequences of the present invention. Exemplary amino acid sequences of these novel polypeptides are set forth in SEQ ID NOs: 2 or 4.

The present invention also encompasses homologues of these polypeptides, such homologues can be at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, or more say 100% homologous to SEQ ID NOs: 2 or 4.

The present invention also encompasses fragments of the above described polypeptides and polypeptides having mutations, such as deletions, insertions or substitutions of one or more amino acids, either naturally occurring or man induced, either randomly or in a targeted fashion.

Amino acid sequence information of the polypeptides of the present invention can be used to generate antibodies, which bind to the polypeptide variants of the present invention.

For example, antibodies may be directed to amino acid sequence coordinates 360-390 of SEQ ID NO: 2. Sequence coordinates 360-390 represent a highly conserved sequence as verified by the multiple comparison analysis (FIG. 6). Due to high sequence homology in this amino acid sequence region, such antibodies are expected to be cross-reactive to other variant polypeptides of the present invention. Accordingly, such antibodies may be useful for identifying novel variant polypeptides of the present invention by immunocloning an expression library of human stromal cells as further described hereinbelow.

Antibodies may also be directed to the unique sequence portions of the polypeptide variants of the present invention (e.g., amino acid coordinates 110-150 of SEQ ID NO: 2 or amino acid coordinates 1-100 of SEQ ID NO: 4) or to unique sequences, which bridge the common portions of the polypeptides of the present and unique sequence regions. Specific peptides chosen for antibody generation are preferably selected immunogenic (i.e., capable of stimulating an antibody response). Parameters for testing peptide immunogenicity are well known in the art including, but not limited to, foreginess, molecular size, chemical composition and heterogeneity and susceptibility to antigen processing and presentation. Various sequence analysis software applications are known in the art, which provide an immunogenicity index according to, for example, the Jameson-Wolf algorithm. Examples include, but are not limited to, Sciprot (available from www.asiaonline.net.hk/˜twcbio/DOCS/1/scPrtein.htm) and Macvector (available from www.accelrys.com/products/macvector/) as well as the widely utilized GCG package (Genetics Computer Group, Wisconsin).

The term “antibody” as used in this invention includes whole antibody molecules as well as functional fragments thereof, such as Fab, F(ab′)₂, and Fv that are capable of binding with antigenic portions of the target polypeptide. These functional antibody fragments constitute preferred embodiments of the present invention, and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule as described in, for example, U.S. Pat. No. 4,946,778.

Methods of generating such antibody fragments are well known in the art. (See for example, Harlow and Lane, Antibodies: A Laboratory Manual, Cold Spring Harbor Laboratory, New York, 1988, incorporated herein by reference).

Purification of serum immunoglobulin antibodies (polyclonal antisera) or reactive portions thereof can be accomplished by a variety of methods known to those of skill in the art including, precipitation by ammonium sulfate or sodium sulfate followed by dialysis against saline, ion exchange chromatography, affinity or immunoaffinity chromatography as well as gel filtration, zone electrophoresis, etc. (see Goding in, Monoclonal Antibodies: Principles and Practice, 2nd ed., pp. 104-126, 1986, Orlando, Fla., Academic Press). Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains. Additional classes include IgD, IgE, IgA, IgM and related proteins.

Methods for the generation and selection of monoclonal antibodies are well known in the art, as summarized for example in reviews such as Tramontano and Schloeder, Methods in Enzymology 178, 551-568, 1989. A recombinant HNRPLL polypeptide (or fragment therof) of the present invention may be used to generate antibodies in vitro. More preferably, the recombinant HNRPLL of the present invention is used to elicit antibodies in vivo. In general, a suitable host animal is immunized with the recombinant HNRPLL of the present invention. Advantageously, the animal host used is a mouse of an inbred strain. Animals are typically immunized with a mixture comprising a solution of the recombinant HNRPLL of the present invention in a physiologically acceptable vehicle, and any suitable adjuvant, which achieves an enhanced immune response to the immunogen. By way of example, the primary immunization conveniently may be accomplished with a mixture of a solution of the recombinant HNRPLL of the present invention and Freund's complete adjuvant, said mixture being prepared in the form of a water in oil emulsion. Typically the immunization will be administered to the animals intramuscularly, intradermally, subcutaneously, intraperitoneally, into the footpads, or by any appropriate route of administration. The immunization schedule of the immunogen may be adapted as required, but customarily involves several subsequent or secondary immunizations using a milder adjuvant such as Freund's incomplete adjuvant. Antibody titers and specificity of binding to the HNRPLL can be determined during the immunization schedule by any convenient method including by way of example radioimmunoassay, or enzyme linked immunosorbant assay, which is known as the ELISA assay. When suitable antibody titers are achieved, antibody-producing lymphocytes from the immunized animals are obtained, and these are cultured, selected and cloned, as is known in the art. Typically, lymphocytes may be obtained in large numbers from the spleens of immunized animals, but they may also be retrieved from the circulation, the lymph nodes or other lymphoid organs. Lymphocytes are then fused with any suitable myeloma cell line, to yield hybridomas, as is well known in the art. Alternatively, lymphocytes may also be stimulated to grow in culture, and may be immortalized by methods known in the art including the exposure of these lymphocytes to a virus, a chemical or a nucleic acid such as an oncogene, according to established protocols. After fusion, the hybridomas are cultured under suitable culture conditions, for example in multi-well plates, and the culture supernatants are screened to identify cultures containing antibodies that recognize the hapten of choice. Hybridomas that secrete antibodies that recognize the recombinant HNRPLL of the present invention are cloned by limiting dilution and expanded, under appropriate culture conditions. Monoclonal antibodies are purified and characterized in terms of immunoglobulin type and binding affinity.

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, in U.S. Pat. Nos. 4,036,945 and 4,331,647, and references contained therein, which patents are hereby incorporated by reference in their entirety (see also Porter, R. R., Biochem. J., 73: 119-126, 1959). Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of V_{H} and V_{L} chains. This association may be noncovalent, as described in Inbar et al. (Proc. Nat'l Acad. Sci. USA 69:2659-62, 1972). Alternatively, the variable chains can be linked by an intermolecular disulfide bond or cross-linked by chemicals such as glutaraldehyde. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, all of which are hereby incorporated, by reference, in entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick and Fry Methods, 2: 106-10, 1991).

Humanized forms of non-human (e.g., murine) antibodies are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues, which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source, which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human monoclonal antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Other stromal RNA regulating factors may be identified using methods which are well known in the art. For example, a human stromal cell cDNA library may be screened using oligonucleotides specific to human SRRF cDNA.

As used herein, the term “oligonucleotide” refers to a single-stranded or double-stranded oligomer or polymer of ribonucleic acid (RNA) or deoxyribonucleic acid (DNA) or mimetics thereof. This term includes oligonucleotides composed of naturally occurring bases, sugars, and covalent internucleoside linkages (e.g., backbone), as well as oligonucleotides having non-naturally occurring portions, which function similarly to respective naturally occurring portions.

Oligonucleotides designed according to the teachings of the present invention can be generated according to any oligonucleotide synthesis method known in the art, such as enzymatic synthesis or solid-phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

The oligonucleotide of the present invention is of at least 17, at least 18, at least 19, at least 20, at least 22, at least 25, at least 30 or at least 40, bases specifically hybridizable with polynucleotide sequences of the present invention.

According to one embodiment of this aspect of the present invention, the oligonucleotides comprise sequences which are present in all known isoforms of SRRF such as sequences which are present in exons 9-11 of the SRRF gene. Preferably, the screening is performed at moderate to stringent hybridization conditions, characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

Alternatively, screening of human cDNA libraries with oligonucleotides corresponding to a specific SRRF splice variant sequences can be carried out at reduced stringency to identify other human SRRF splice variant cDNAs. Examples of such oligonucleotides include, but are not limited to those that are capable of specifically hybridizing to an isolated polynucleotide as set forth in SEQ ID NO:1 and not to an isolated polynucleotide as set forth in SEQ ID NO: 3. Other examples of such oligonucleotides include, but are not limited to those that are capable of specifically hybridizing to an isolated polynucleotide as set forth in SEQ ID NO:3 and not to an isolated polynucleotide as set forth in SEQ ID NO: 1. It will be appreciated that the oligonucleotides may also hybridize to bridging regions which are comprised in the new variants.

As used herein, the phrase “capable of hybridizing” refers to forming a double strand molecule such as RNA:RNA, RNA:DNA and/or DNA:DNA molecules.

Alternatively, or additionally, novel stromal RNA regulating factors may be identified by immunocloning as mentioned hereinabove. Essentially, an expression library of human stromal cells (commercially available at Stratagene, La Jolla, Calif.) may be screened using an antibody raised against an SRRF polypeptide. The procedure of immunocloning is described in Example 1 hereinbelow. Methods of generating antibodies to SRRF polypeptides are described hereinbelow.

Yet alternatively, or additionally, computer programs are available in the art that identify polypeptide variants based on genomic sequences. Genomic-based polypeptide variant identification programs include FgenesH (A. Salamov and V. Solovyev, “Ab initio gene finding in Drosophila genomic DNA,” Genome Research. 2000 April; 10(4):516-22); Grail (URL compbio.oml.gov/Grail-bin/-EmptyGrailForm) and GenScan (URL genes.mit.edu/GENSCAN.html). Electronic northerns may be performed to confirm that the novel polypeptide variants are specifically expressed in mesenchymal stem cells.

Following identification, the polynucleotides can be further analyzed using a variety of techniques available in the art, such as full-length cloning, proteomic validation, PCR-based validation, and 5′ RACE validation, etc. Specific expression of such identified polynucleotides in mesenchymal stem cells may be confirmed at the RNA level or the protein level as further described hereinbelow.

As mentioned, the polynucleotides and polypeptides of the present invention are specifically expressed in mesenchymal stem cells. Thus, as shown in Table 4 of Example 1 of the Examples section hereinbelow, Transcript A and Transcript B are expressed in mesenchymal stem cells and not in skeletal muscle, brain, heart, kidney, liver, pancreas, lung and placenta. The polynucleotides and polypeptides of the present invention may therefore be used as mesenchymal stem cell markers.

Thus, according to another aspect of the present invention there is provided a method of identifying a mesenchymal stem cell comprising identifying in a biological sample a cell expressing the polynucleotides or the polypeptides of the present invention.

As used herein the phrase “biological sample” refers to a sample of tissue or fluid isolated from an individual which comprises bone marrow cells, including, but not limited to, for example, bone marrow, plasma, serum, spinal fluid, lymph fluid, the external sections of the skin, respiratory, intestinal, and genitourinary tracts, tears, saliva, milk, blood cells, synovial cell fluid, tumors, organs such as synovial tissue and also samples of in vivo cell culture constituents (e.g., synovial fluid cells). Preferably, the biological sample is a bone marrow sample. Bone marrow is typically aspirated surgically using methods known in the art.

Following is a non-limiting list of methods which be used to identify mesenchymal stem cells by detecting the polynucleotides of the present invention.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, calorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell 1 LCM system available from Arcturus Engineering (Mountainview, Calif.).

Oligonucleotide microarray—In this method oligonucleotide probes capable of specifically hybridizing with the polynucleotides of the present invention are attached to a solid surface (e.g., a glass wafer). Each oligonucleotide probe is of approximately 20-25 nucleic acids in length. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

As mentioned hereinabove, mesenchymal stem cells may be identified by detecting the polypeptides of the present invention. Following is a non-limiting list of methods which be used to identify mesenchymal stem cells by detecting the polypeptides of the present invention.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a calorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

In vitro activity assays: In these methods the activity of a particular enzyme is measured in a protein mixture extracted from the cells. The activity can be measured in a spectrophotometer well using colorimetric methods or can be measured in a non-denaturing acrylamide gel (i.e., activity gel). Following electrophoresis the gel is soaked in a solution containing a substrate and calorimetric reagents. The resulting stained band corresponds to the enzymatic activity of the protein of interest. If well calibrated and within the linear range of response, the amount of enzyme present in the sample is proportional to the amount of color produced. An enzyme standard is generally employed to improve quantitative accuracy.

As mentioned herein above, the present inventor has shown that there is a very high sequence homology between SRRF isoforms and other hnRNP proteins (FIG. 6). Since hnRNPs are known to play an extensive role in RNA processing, the present inventor has postulated that the polynucleotides and/or polypeptides of the present invention may be used to modulate RNA processing in mesenchymal stem cells. By modulating RNA processing in the mesenchymal stem cell, specific differentiation pathways may be triggered and thus the polynucleotides and/or polypeptides of the present invention may play a role in directing differentiation of mesenchymal stem cells.

Thus, according to yet another aspect of the present invention there is provided a method of modulating RNA processing in mesenchymal cells. The method is affected by contacting the mesenchymal stem cells with an agent capable of regulating an expression and/or activity of the isolated polypeptides of the present invention.

As used herein, the phrase “RNA processing” refers to any reaction that results in covalent modification of an RNA sequence. Accordingly, the phrase “RNA processing” may incorporate such procedures as 5′-cap formation, methylation, 3′-end cleavage, poly-adenylation and splicing of introns. Since RNA export from the nucleus is controlled together with splicing, transportation of mRNA may also be considered a form of RNA processing.

According to this aspect of the present invention, modulating RNA processing in mesenchymal cells may be effected both in vivo and/or ex vivo.

As mentioned, the method of this aspect of the present invention is affected by contacting the mesenchymal stem cells with an agent capable of regulating an isolated polypeptide of the present invention.

The term “regulating” as used herein refers to both upregulating (i.e., increasing) or downregulating (i.e., decreasing) a polypeptide of the present invention.

It will be appreciated that modulating RNA processing may be effected by regulating one particular isoform of SRRF or any combination of isoforms of SRRF. Accordingly, the agents of the present invention may be directed against a unique sequence present in the SRRF isoform or polynucleotide encoding same or may be directed against a sequence common to a group of SRRF isoforms or polynucleotides encoding same.

Agents capable of upregulating the polypeptides of the present invention may comprise the isolated polynucleotides of the present invention.

Such polynucleotide sequences are typically inserted into expression vectors to enable expression of the recombinant polypeptide. The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Recombinant viral vectors may also be used to synthesize the polynucleotides of the present invention. Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Bone marrow cells can be targeted using the human T cell leukemia virus type I (HTLV-I).

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Downregulating the function of the polypeptide of the present invention can be effected at the RNA level or at the protein level. According to one embodiment of this aspect of the present invention the agent is an oligonucleotide capable of specifically hybridizing (e.g., in cells under physiological conditions) to a particular polynucleotide of the present invention (e.g. the polynucleotide as set forth in SEQ ID NO:1 or SEQ ID NO:3). Such oligonucleotides have been described hereinabove.

The prior art teaches of a number of delivery strategies which can be used to efficiently deliver oligonucleotides into a wide variety of cell types [see, for example, Luft J Mol Med 76: 75-6 (1998); Kronenwett et al., Blood 91: 852-62 (1998); Rajur et al., Bioconjug Chem 8: 935-40 (1997); Lavigne et al., Biochem Biophys Res Commun 237: 566-71 (1997) and Aoki et al., (1997) Biochem Biophys Res Commun 231: 540-5 (1997)].

A small interfering RNA (siRNA) molecule is another example of an agent capable of downregulating the expression of a polypeptide of the present invention. RNA interference is a two-step process. During the first step, which is termed the initiation step, input dsRNA is digested into 21-23 nucleotide (nt) small interfering RNAs (siRNA), probably by the action of Dicer, a member of the RNase III family of dsRNA-specific ribonucleases, which cleaves dsRNA (introduced directly or via an expressing vector, cassette or virus) in an ATP-dependent manner. Successive cleavage events degrade the RNA to 19-21 bp duplexes (siRNA), each strand with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al., (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs, which would generate more siRNAs, or by replication of the siRNAs formed. Alternatively or additionally, amplification could be effected by multiple turnover events of the RISC [Hammond et al., Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the SRRF polynucleotide sequence target is scanned downstream for AA dinucleotide sequences. Occurrence of each AA and the 3′ adjacent 19 nucleotides is recorded as potential siRNA target sites.

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites that exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating the expression of a polypeptide of the present invention is a DNAzyme molecule capable of specifically cleaving its encoding polynucleotide. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;94:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of Chronic Myelogenous Leukemia (CML) and Acute Lymphocytic Leukemia (ALL).

Another agent capable of downregulating the expression of the polypeptide of the present invention is a ribozyme molecule capable of specifically cleaving its encoding polynucleotide. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications.

An additional method of downregulating the function of the SRRF of the present invention is via triplex forming oligonuclotides (TFOs). In the last decade, studies have shown that TFOs can be designed which can recognize and bind to polypurine/polypirimidine regions in double-stranded helical DNA in a sequence-specific manner. Thus the DNA sequence encoding the polypeptide of the present invention can be targeted thereby down-regulating the polypeptide.

The recognition rules governing TFOs are outlined by Maher III, L. J., et al., Science (1989) 245:725-730; Moser, H. E., et al., Science (1987)238:645-630; Beal, P. A., et al., Science (1991) 251:1360-1363; Cooney, M., et al., Science(1988)241:456-459; and Hogan, M. E., et al., EP Publication 375408. Modification of the oligonuclotides, such as the introduction of intercalators and backbone substitutions, and optimization of binding conditions (pH and cation concentration) have aided in overcoming inherent obstacles to TFO activity such as charge repulsion and instability, and it was recently shown that synthetic oligonucleotides can be targeted to specific sequences (for a recent review see Seidman and Glazer (2003) J Clin Invest;112:487-94).

In general, the triplex-forming oligonucleotide has the sequence correspondence:

| | | | | |
|---|---|---|---|---|
| oligo | 3′--A | G | G | T |
| duplex | 5′--A | G | C | T |
| duplex | 3′--T | C | G | A |

However, it has been shown that the A-AT and G-GC triplets have the greatest triple helical stability (Reither and Jeltsch (2002), BMC Biochem,, Sept12, Epub). The same authors have demonstrated that TFOs designed according to the A-AT and G-GC rule do not form non-specific triplexes, indicating that the triplex formation is indeed sequence specific.

Thus for any given sequence in the regulatory region a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and subsequent formation of the triple helical structure with the target DNA, induces steric and functional changes, blocking transcription initiation and elongation, allowing the introduction of desired sequence changes in the endogenous DNA and results in the specific downregulation of gene expression. Examples of such suppression of gene expression in cells treated with TFOs include knockout of episomal supFG1 and endogenous HPRT genes in mammalian cells (Vasquez et al., Nucl Acids Res. (1999) 27:1176-81, and Puri, et al., J Biol Chem, (2001) 276:28991-98), and the sequence- and target-specific downregulation of expression of the Ets2 transcription factor, important in prostate cancer etiology (Carbone, et al., Nucl Acid Res. (2003) 31:833-43), and the pro-inflammatory ICAM-1 gene (Besch et al., J Biol Chem, (2002) 277:32473-79). In addition, Vuyisich and Beal have recently shown that sequence specific TFOs can bind to dsRNA, inhibiting activity of dsRNA-dependent enzymes such as RNA-dependent kinases (Vuyisich and Beal, Nuc. Acids Res (2000);28:2369-74).

Additionally, TFOs designed according to the abovementioned principles can induce directed mutagenesis capable of effecting DNA repair, thus providing both downregulation and upregulation of expression of endogenous genes [Seidman and Glazer, J Clin Invest (2003) 112:487-94]. Detailed description of the design, synthesis and administration of effective TFOs can be found in U.S. patent application Ser. Nos. 2003 017068 and 2003 0096980 to Froehler et al., and 2002 0128218 and 2002 0123476 to Emanuele et al., and U.S. Pat. No. 5,721,138 to Lawn.

As mentioned hereinabove, down regulating the function of the polypeptide of the present invention can also be affected at the protein level.

Thus, another example of an agent capable of downregulating a polypeptide of the present invention is an antibody or antibody fragment capable of specifically binding an SRRF or a particular isoform thereof, preferably to its active site, thereby preventing its function. Methods of producing such antibodies are described hereinabove.

Regardless of the agents employed the effect of same on mesenchymal stem cells may be determined using well known molecular biology, biochemical or cell biology techniques. The specific assay will be selected according to the particular researchers needs and expertise.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Cloning and Analysis of Bone Marrow Stromal Cell (MSC) Specific cDNAs

In order to identify polypeptides of interest, an antibody raised against a marrow stromal osteogenic cell line (MBA-15) was used to immunoclone polypeptides from a human stromal cell expression library.

Materials and Methods

Cloning: An expression library of human stromal cells (Stratagene, La Jolla, Calif.) was plated on LB-ampicillin agar plates and induced with isopropyl D-1-thiogalactopyranoside (IPTG, Sigma, USA) for secretion of fusion protein that was captured onto nitrocellulose. For cloning, the monoclonal antibody, MMS-85 (Benayahu et al, 1995, J Bone Miner Res 10, 1496-503) was reacted with the proteins captured on the nitrocellulose. The colonies that were the most highly expressed were selected. The cDNAs were digested from BlueScript (BS) plasmid with EcoRI and XhoI, run on a gel, blotted to a nylon membrane (Bio Rad, USA) and analyzed by Southern blot. Probes were labeled with [³²P]αCTP by random prime (Stratagene, USA) and used for hybridization overnight at 65° C. The membranes were subsequently exposed to X-OMAT AR film (Kodak, USA). The clones were sequenced by universal T3, T7 or cDNA specific primers in both directions on the double-stranded BS plasmid using Applied Bio-systems (Sequencing Unit, Tel Aviv University).

Northern Blot: Northern blot was used to analyze the expression of the two mRNA isoforms in MSC. Total RNA was separated in formaldehyde gels and blotted to a nylon membrane (Bio Rad, USA). Probes were labeled with [³²P]αCTP by random prime (Stratagene, USA) and used for hybridization overnight at 65° C. The membranes were subsequently exposed to X-OMAT AR film (Kodak, USA).

RT-PCR: To analyze tissue distribution primers specific for amplification of alternatively spliced transcripts were used as set forth in Table 1.

TABLE 1

| | | | Expected |
|---|---|---|---|
| primers | Sequence | SEQ ID NO: | size |
| G3PDH-F | ACCACAGTCCATGCCATCAC | SEQ ID NO: 5 | 445 bp |
| G3PDH-R | TCCACCACCCTGTTGCTGTA | SEQ ID NO: 6 | |
| SRRF-1 | ACAAAGTTCTTCTGCTC | SEQ ID NO: 7 | 580 bp, |
| | | | 682 bp, |
| | | | 765 bp |
| SRRF-2 | ATTAAGGTGTGTGACAGC | SEQ ID NO: 8 | |
| SRRF-3 | CAGATTCTCTGAAGCCCACC | SEQ ID NO: 9 | 1246 bp |
| SRRF-4 | CATTCCTGGTACAGCACTGGT | SEQ ID NO: 10 | |

cDNA was prepared from cultured MSC and cDNA from various tissues (Clontech Quick screen library™ kit). RNA was extracted from cultured marrow stromal cells (MSC) with EZ RNA kit (Biological industries, Bet-Haemek, Israel) and reverse transcribed (RT) using avian myeloblastosis virus reverse transcriptase (AMV-RT) and oligo-dT. The cDNA then served as a template for PCR (Takara Shuzo Co. Ltd., Japan). The integrity of the RNA, the efficiency of RT reaction and the quality of cDNA subjected to the RT-PCR was controlled by amplification of transcript of Glucose-3-Phosphate Dehydrogenase (G3PDH).

Bioinformatic analysis of SRRF gene and protein: The bioinformatic analysis at the gene level included genomic analysis, analysis of the translation initiation site, termination signals and UTR. Web-based programs used for the analysis of SRRF DNA are summarized in Table 2 hereinbelow.

TABLE 2

| Web site | Analysis |
|---|---|
| http://www.ncbi.nlm.nih.gov | NCBI (BLAST, CD- |
| | search, UniGene, |
| | OMIM, Locus Link,) |
| http://genes.mit.edu/GENSCAN.html | GENSCAN |
| www.expasy.ch/tools/dna.html | Translation, ORF |
| www.expasy.ch/cgi-bin/protparam | Protein parameters |
| Coot.embl-heidelberg.de/SMART | Functional & Structural |
| | domains |
| www2.ebi.ac.uk/ppsearch/ | Protein Motifs |
| www.pbil.ibcp.fr/cgi- | |
| bin/npsa_automat.pl?page=npsa_prosite.html | |
| http://protein.toulouse.inra.fr/multalin/cgi- | Multalin (hierarchical |
| bin/multalin.pl | clustering and multiple |
| | sequence alignment) |

Results

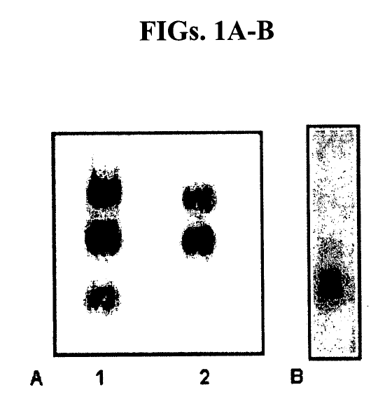

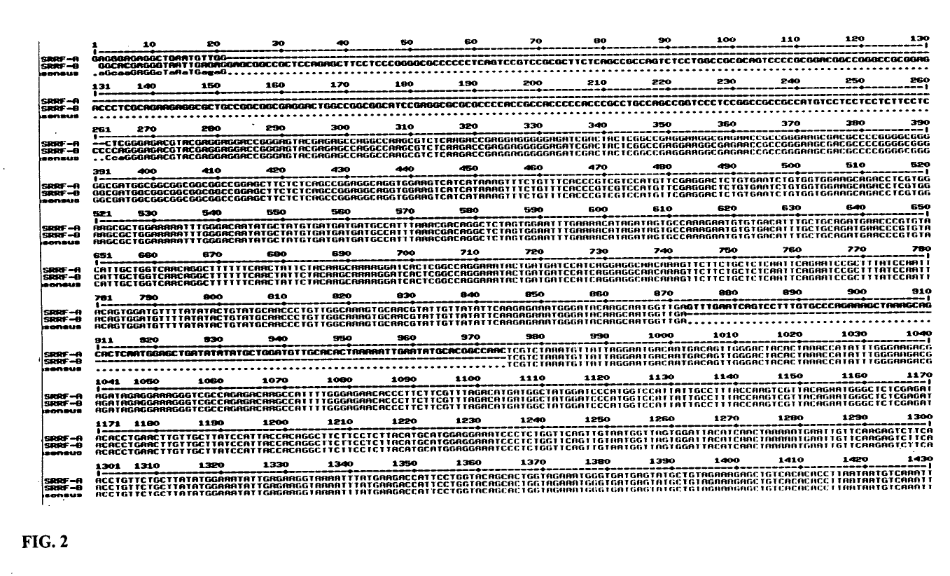

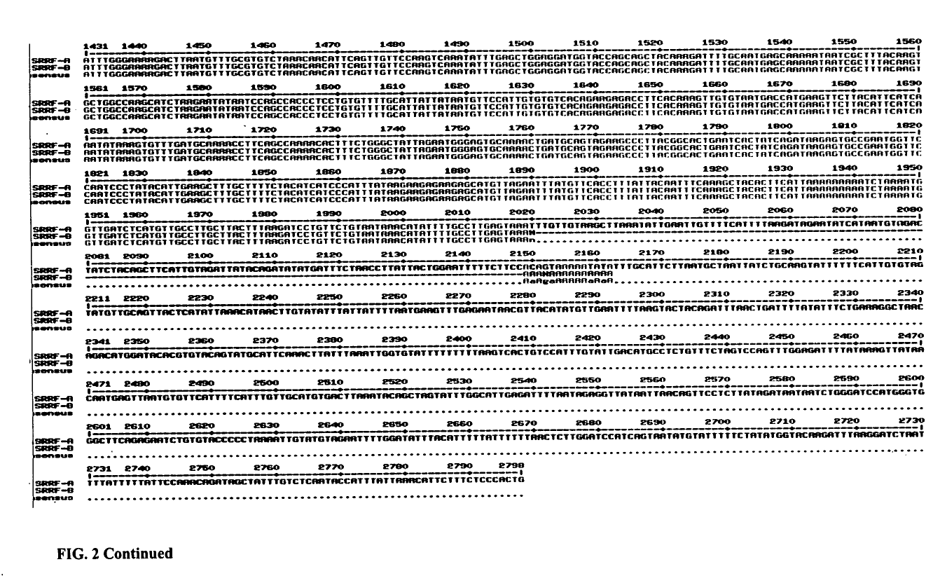

Cloning and gene structure: Two cDNAs—transcripts A and B (SEQ ID NO:1 and SEQ ID NO:3, respectively) were immuno-cloned from the human stromal cell library. The two cDNAs were digested with restriction enzymes XhoI and EcoR I from the BS vector and the generated fragments of different sizes were confirmed for homology by Southern blot (FIG. 1A). The mRNA expression of the two transcripts in MSC was verified by Northern blot (FIG. 1B). Sequencing of two cDNAs demonstrated 100% homology over a 1454-bp region which confirmed that these clones were derived from the same gene. The cDNAs represented two transcripts of different size: transcript-A consisted of 2557 bp and transcript-B consisted of 1931 bp. FIG. 2 shows a sequence alignment scheme of the two isoforms that are different in three regions: (i) Alignment between nucleotide 25 of transcript A starts from nucleotide 265 of transcript B; (ii) transcript A has an insertion of 103 bp after nucleotide 871 of transcript B; (iii) and extends by 779 bp at the 3′ prime end. The alignment suggests that both transcripts are alternatively spliced forms that possess different ORFs, based on exclusion of alternatively spliced exon, and different 3′ UTRs.

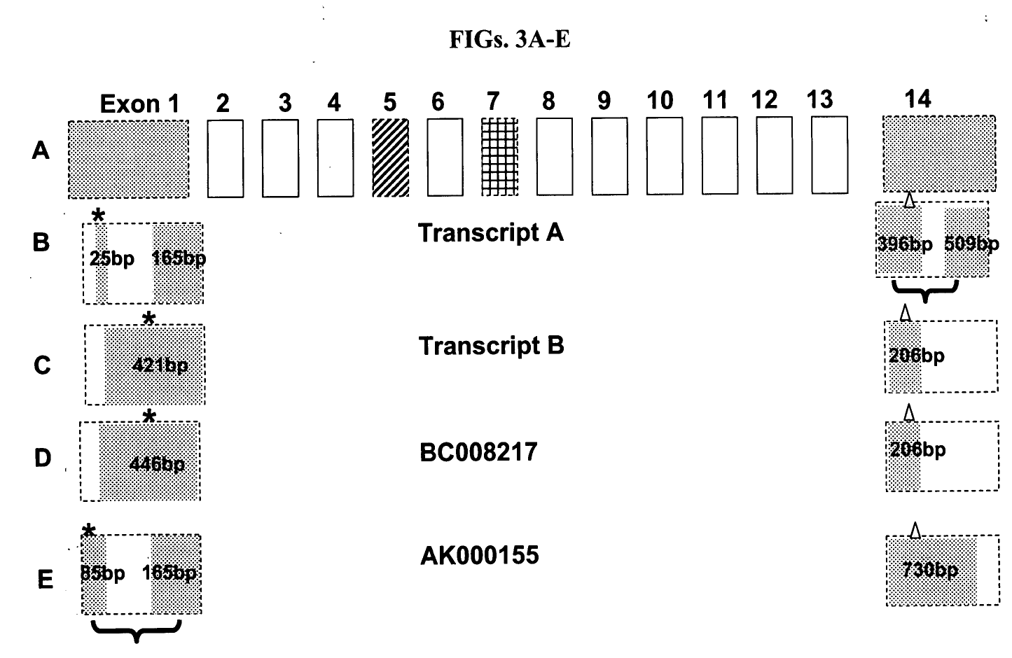

The gene coding for transcript A and B (referred to herein as the stromal RNA regulating factor (SRRF) gene) is aligned to genome locus at 2p22 region. To resolve the complete gene structure transcripts A and B were aligned against NCBI NR database to find homology to other cDNAs. Three cDNAs [AK000155—SEQ ID NO:11, AL512692—SEQ ID NO:12 and BC017480—SEQ ID NO:13] derived from the same genomic region representing additional alternatively spliced variants were identified. These spliced variants were expressed by different cell types: colon (AK000155), lymph node (AL512692), placenta and neuroblastoma (BC017480). Alignment of the three transcripts [AK000155, AL512692 and BC017480] along with transcripts A and B against genome sequence enabled the elucidation of the complete exon/intron structure of the SRRF gene (FIGS. 3a-e, 4a-c and Table 3 hereinbelow).

TABLE 3

| | total | | | | | |
|---|---|---|---|---|---|---|
| | exon | | | intermediate | | |
| transcript | number | first exon | | exons | last exon | |
| Transcript A | 14 | 25 bp | 165 bp | 2-6, 8-13 | 396 bp | 590 |
| | | | | | | bp |

| Transcript B | 12 | 421 bp | | 2-4, 6, 8-13 | 206 bp |
|---|---|---|---|---|---|
| AK000155 | 14 | 85 bp | 165 bp | 2-6, 8-13 | 730 bp |

| | | | | |
|---|---|---|---|---|
| BC017480 | 13 | 446 bp | 2-6, 8-13 | 206 bp |
| AL512692 | 13 | missing | 2-13 | 730 bp |

The gene consists of 14 exons with sizes ranging from 60 bp to 730 bp (FIGS. 3a-e, 4b-c). Comparison between analyzed transcripts revealed that Transcript B and BC017480 start from exon 1 but differ in their 5′ UTRs region (FIGS. 3a-e, Table 3). The first exon of transcript AK000155 is spliced into two (85 bp and 165 bp) separated by 255 bp, thus the ORF begins differently. AL512692 starts from exon 2; Transcript B is missing exon 5. Exon 7 is expressed only by mRNA AL512692 detected from lymphoid tissue (FIG. 3, Table 3 and 4). This exon was expressed neither by other transcripts found in the Gene Bank, nor by cloned transcripts A and B. Exon 14 is expressed by all transcripts and includes the stop codon and extended 3′ UTR. Three patterns of expression of exon 14 were detected (FIG. 3, Table 3): two transcripts (AK000155/AL512692) have complete exon length of 730 bp, including 565 bp after the stop codon. Transcripts B and BC017480 have a short exon of 206 bp, including 41 bp 3′ UTR. In transcript A, exon 14 is spliced into two parts of 396 and 590 bp and the 68 bp is missing between them. The translated region is 165 bp to the stop codon and the extended part of 821 bp is 3′ UTR (FIG. 3, Table 3).

Differential expression of MSC-derived SRRF transcripts: The uniqueness of MSC specific expression of transcripts A and B was demonstrated by differential expression analysis using cDNA from various tissues. Primers SRRF-1/SRRF-2 (SEQ ID NOs: 4 and 5 respectively) were used to screen cDNA from various tissues by amplifying exons 3-8. The position of the primers on the gene is illustrated in FIGS. 3A-E. The results of the PCR analysis of differential expression for alternatively spliced forms are summarized in Table 4 hereinbelow.

TABLE 4

| cDNA | SRRF-1 & SRRF-2 | | | SRRF-3 & | |
|---|---|---|---|---|---|
| Expression | AK000155/ | Transcript | | SRRF-4 | GAP- |
| Library | BC017480 | B | AL512692 | Transcript A | DH |
| MSC | + | + | − | + | + |
| Skeletal | + | − | − | − | + |
| muscle | | | | | |
| Brain | − | − | − | − | + |
| Heart | + | − | − | − | + |
| Kidney | + | − | − | − | + |
| Liver | − | − | − | − | + |
| Pancreas | + | − | − | − | + |
| Lung | + | − | − | − | + |
| Placenta | + | − | − | − | + |
| Universal | + | − | − | − | + |

As seen in FIG. 4B, the PCR amplification of MSC cDNA resulted in two bands of 682 bp and 580 bp, corresponding to the cDNA missing exon 7, and the cDNA missing both exons 5 and 7. The lower band of 580 bp corresponds to transcript B missing exon 5 and 7 that is uniquely expressed in MSC and not expressed in the universal library or in other cDNAs from any tissue studied (FIG. 4B, Table 4). The 682 bp PCR product was amplified from skeletal and heart muscle, placenta, pancreas, kidney and lung tissues, and absent in liver and brain (FIG. 4B, Table 4). PCR product corresponding to AL512692 cDNA that expressed exon 7 was expected as 765 bp; however, it was undetectable in any of the studied expression libraries and is specific for lymphoid tissue (FIG. 4B, Table 4). Transcript B is the only spliced form missing both exons 5 and 7 (FIG. 4B, Table 4). The splicing pathways of the 3′ UTR region were analyzed by SRRF-3/SRRF-4 primers (SEQ ID NOs: 9 and 10 respectively) revealing specific 3′UTR expression for transcript A that was detected only in MSC-cDNA and not in any other tissue studied (FIG. 4C, Table 4).

In summary, the gene is encoded by 14 exons in which exons 5 and 7 are alternatively spliced. This gene also possesses alternatively spliced forms that differ in the 3′ and 5′ UTR regions. The transcripts A and B are uniquely expressed by the MSC library. Transcript A is missing part of a sequence from the middle of the 3′ UTR region, and transcript B lacks exon 5, while both transcripts do not express exon 7. No analogs of this pattern of expression were found in the EST data base.

### Example 2

### In Silico Translation of Transcript-A and Transcript-B

Materials and Methods

The ExPASy (www.expasy.ch/tools/dna.html) tool was used to in silico translate the two alternatively spliced isoforms.

The bioinformatic analysis at the protein level included analysis of the physical properties based on sequence, sequence statistics, specialized features such as—coiled coils, transmembrane regions, low complexity regions and protein domains, and gene family analysis. Web-based programs used for the analysis of SRRF translated polypeptides are summarized in Table 2 hereinabove.

Results

Transcript-A possessed an ORF of 1314 bp translated to a 437-AA polypeptide (SEQ ID NO: 2). The ORF of Transcript-B is 1526 bp that is translated to a 508-AA polypeptide (SEQ ID NO: 4). Analysis of domains of these polypeptides demonstrated that they shared two RNA binding motif (RRM) domains. A third RRM was found only in polypeptide-A (SEQ ID NO: 2), which is encoded by the unique exon 5 (FIG. 5, Table 3).

A summary of the protein features in the predicted polypeptides is set forth in Table 5 hereinbelow.

TABLE 5

| | Transcript-A | Transcript-B | Program |
|---|---|---|---|
| mRNA Length | 2335 bp | 1931 bp | |
| ORF Length | 1314 bp (437 aa) | 1527 bp (508 aa) | ORF finder |
| | | | (NCBI) |
| ORF MW | 48791 Da | 56467 Da | Expasy |
| ORF PI | 8.95 | 7.56 | Expasy |
| Total Number of negatively | 39 | 57 | Expasy |
| charged residues (Asp + Glu) | | | |
| Total Number of positively | 50 | 58 | Expasy |
| charged residues (Arg + Lys) | | | |
| Protein domains | 3 rrm motifs | 2 rrm motifs | Pfam, SMART, |
| | PTB(1), SH2(5), | DNA binding motif | Prints, Psort, |
| | WW(1) | Coiled coil region | prosite |
| | | 14-3-3(1), | |
| | | PDZ(1), PTB(1), | |
| | | SH2(5), WW (4) | |
| PolyA Signal | Yes | Yes | GENSCAN |
| PolyA Tail | No | Yes | |

Polypeptide-A contains several protein-protein interaction motifs (PTB, SH2 and WW) (FIG. 5, Table 5). Polypeptide-B possesses coiled-coil region, DNA binding domain (ps00043) and other protein-protein interaction motifs (14-3-3 and PDZ) (FIG. 5, Table 5). These proteins are predicted to possess different functions in RNA regulation in the cell. Alignment of SRRF transcripts A and B against the NCBI protein database revealed their homology with the other members of the RRM RNA-binding proteins family. Homology to hnRNP-L protein was detected at 69% and to hnRNP-1 and ROD1 proteins at 46% (FIG. 6).

Conclusion

The results presented in this study, suggest that the SRRF gene encodes a number of isoforms which are members of the RRM subfamily of proteins. The two isoforms identified in this study are MSC-specific. Significant differences were found in the structure and domain composition between the alternatively spliced forms suggesting differences in function of the SRRF proteins. Variability of the expressed domains, ligand motifs and the resulting protein properties were demonstrated. Taking into consideration the restricted expression of transcript A and B in MSC, it is possible that they have an overlapping but independent role in regulating RNA processing in the MSC, and that changes in their ratio may alter alternative splicing events in a tissue-specific manner.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

What is claimed is:

1. An isolated polynucleotide comprising a nucleic acid sequence encoding an HNRPLL polypeptide, said polypeptide being specifically expressed in mesenchymal stem cells and not in skeletal muscle cells.

2. The isolated polynucleotide of claim 1, wherein said nucleic acid sequence does not comprise SEQ ID NO:14.

3. The isolated polynucleotide of claim 1, wherein said nucleic acid sequence does not comprise SEQ ID NO:15.

4. The isolated polynucleotide of claim 1, wherein said nucleic acid sequence does not comprise SEQ ID NO:16.

5. The isolated polynucleotide of claim 1, wherein said nucleic acid sequence comprises a nucleic acid segment as set forth in SEQ ID NO:17 at the 3′ terminus of the isolated polynucleotide.

6. The isolated polynucleotide of claim 1, wherein said nucleic acid sequence comprises a nucleic acid segment as set forth in SEQ ID NO:18.

7. The isolated polynucleotide of claim 3, wherein said nucleic acid sequence is as set forth in SEQ ID NO: 1.

8. The isolated polynucleotide of claim 5, wherein said nucleic acid sequence is as set forth in SEQ ID NO: 1.

9. The isolated polynucleotide of claim 4, wherein said nucleic acid sequence is as set forth in SEQ ID NO: 3.

10. The isolated polynucleotide of claim 6, wherein said nucleic acid sequence is as set forth in SEQ ID NO: 3.

11. The isolated polynucleotide of claim 1, wherein said nucleic acid sequence is as set forth in SEQ ID NO: 1 or SEQ ID NO: 3.

12. An isolated polynucleotide as set forth in SEQ ID NO: 1.

13. An isolated polynucleotide as set forth in SEQ ID NO: 3.

14. An oligonucleotide capable of specifically hybridizing to an isolated polynucleotide as set forth in SEQ ID NO: 1 and not to an isolated polynucleotide as set forth in SEQ ID NO: 3.

15. An oligonucleotide capable of specifically hybridizing to the isolated polynucleotide as set forth in SEQ ID NO: 3 and not to the isolated polynucleotide as set forth in SEQ ID NO: 1.

16. An isolated polypeptide comprising an amino acid sequence encoding an HNRPLL polypeptide, said polypeptide being specifically expressed in mesenchymal stem cells and not in skeletal muscle cells.

17. The isolated polypeptide of claim 16, wherein said amino acid sequence is as set forth in SEQ ID NO: 2 or SEQ ID NO: 4.

18. An isolated polypeptide as set forth in SEQ ID NO: 2.

19. An isolated polypeptide as set forth in SEQ ID NO: 4.

20. An antibody capable of specifically recognizing an isolated polypeptide as set forth in SEQ ID NO: 2 and not an isolated polypeptide as set forth in SEQ ID NO: 4.

21. An antibody capable of specifically recognizing an isolated polypeptide of as set forth in SEQ ID NO: 4 and not an isolated polypeptide as set forth in SEQ ID NO:2.

22. A method of modulating RNA processing in mesenchymal stem cells, comprising contacting the mesenchymal stem cells with an agent capable of regulating an expression and/or activity of the isolated polypeptide of claim 16, thereby regulating RNA processing in mesenchymal stem cells.

23. The method of claim 22, wherein said regulating is down-regulating.

24. The method of claim 23, wherein said agent is selected from the group consisting of an antibody, an antisense, an siRNA, a ribozyme and a DNAzyme.

25. The method of claim 23, wherein said agent comprises An oligonucleotide capable of specifically hybridizing to an isolated polynucleotide as set forth in SEQ ID NO: 1 and not to an isolated polynucleotide as set forth in SEQ ID NO: 3.

26. The method of claim 23, wherein said agent comprises an oligonucleotide capable of specifically hybridizing to the isolated polynucleotide as set forth in SEQ ID NO: 3 and not to the isolated polynucleotide as set forth in SEQ ID NO: 1.

27. The method of claim 24, wherein said antibody is capable of specifically recognizing an isolated polypeptide as set forth in SEQ ID NO: 2 and not an isolated polypeptide as set forth in SEQ ID NO: 4.

28. The method of claim 24, wherein said antibody is capable of specifically recognizing an isolated polypeptide of as set forth in SEQ ID NO: 4 and not an isolated polypeptide as set forth in SEQ ID NO: 2.

29. The method of claim 22, wherein said regulating is up-regulating.

30. The method of claim 29, wherein said agent is the isolated polynucleotide which comprises a nucleic acid sequence encoding an HNRPLL polypeptide, said polypeptide being specifically expressed in mesenchymal stem cells and not in skeletal muscle cells.

31. A method of identifying a mesenchymal stem cell comprising identifying in a biological sample a cell expressing the polynucleotide of claim 1, said cell being the mesenchymal stem cell.

32. The method of claim 31, wherein said identifying is effected via an oligonucleotide capable of specifically hybridizing to an isolated polynucleotide as set forth in SEQ ID NO: 1 and not to an isolated polynucleotide as set forth in SEQ ID NO: 3.

33. The method of claim 31, wherein said identifying is effected via an oligonucleotide capable of specifically hybridizing to the isolated polynucleotide as set forth in SEQ ID NO: 3 and not to the isolated polynucleotide as set forth in SEQ ID NO: 1.

34. A method of identifying a mesenchymal stem cell comprising identifying in a biological sample a cell expressing a polypeptide which comprises an amino acid sequence encoding an HNRPLL polypeptide, said polypeptide being specifically expressed in mesenchymal stem cells and not in skeletal muscle cells, said cell being the mesenchymal stem cell.

35. The method of claim 34, wherein said identifying is effected via an antibody capable of specifically recognizing an isolated polypeptide as set forth in SEQ ID NO: 2 and not an isolated polypeptide as set forth in SEQ ID NO: 4.

36. The method of claim 34, wherein said identifying is effected via an antibody capable of specifically recognizing an isolated polypeptide as set forth in SEQ ID NO: 2 and not an isolated polypeptide as set forth in SEQ ID NO: 4.


SEQUENCE LISTING (18 sequences)

SEQ ID NO: 1 - DNA, 2557 bp, Homo sapiens
gagggagaggctgaatgttggctcgggagacgtacgaggaggaccgggagtacgagagccaggccaagcgtctcaagaccgaggagggggagatcgactactcggccgaggaaggcgagaaccgccgggaagcgacgccccggggcgggggcgatggcggcggcggcggccggagcttctctcagccggaggcaggtggaagtcatcataaagtttctgtttcacccgtcgtccatgttcgaggactctgtgaatctgtggtggaagcagacctcgtggaagcgctggaaaaatttgggacaatatgctatgtgatgatgatgccatttaaacgacaggctctagtggaatttgaaaacatagatagtgccaaagaatgtgtgacatttgctgcagatgaacccgtgtacattgctggtcaacaggcttttttcaactattctacaagcaaaaggatcactcggccaggaaatactgatgatccatcaggaggcaacaaagttcttctgctctcaattcagaatccgctttatccaattacagtggatgttttatatactgtatgcaaccctgttggcaaagtgcaacgtattgttatattcaagagaaatgggatacaagcaatggttgagtttgaatcagtcctttgtgcccagaaagctaaagcagcactcaatggagctgatatatatgctggatgttgcacactaaaaattgaatatgcacggccaactcgtctaaatgttattaggaatgacaatgacagttgggactacactaaaccatatttgggaagacgagatagaggaaagggtcgccagagacaagccattttgggagaacacccttcttcgtttagacatgatggctatggatcccatggtccattattgcctttaccaagtcgttacagaatgggctctcgagatacacctgaacttgttgcttatccattaccacaggcttcttcctcttacatgcatggaggaaatccctctggttcagttgtaatggttagtggattacatcaactaaaaatgaattgttcaagagtcttcaacctgttctgcttatatggaaatattgagaaggtaaaatttatgaagaccattcctggtacagcactggtagaaatgggtgatgagtatgctgtagaaagagctgtcacacaccttaataatgtcaaattatttgggaaaagacttaatgtttgcgtgtctaaacaacattcagttgttccaagtcaaatatttgagctggaggatggtaccagcagctacaaagattttgcaatgagcaaaaataatcgctttacaagtgctggccaagcatctaagaatataatccagccaccctcctgtgttttgcattattataatgttccattgtgtgtcacagaagagaccttcacaaagttgtgtaatgaccatgaagttcttacattcatcaaatataaagtgtttgatgcaaaaccttcagccaaaacactttctgggctattagaatgggagtgcaaaactgatgcagtagaagcccttacggcactgaatcactatcagataagagtgccgaatggttccaatccctatacattgaagctttgcttttctacatcatcccatttataagaagagaagagcatgttagaatttatgttcacctttattacaatttcaaagctacacttcattaaaaaaaaatctaaaatggttgatctcatgttgccttgcttactttaagatcctgttctgtaataaacatattttgccttgagtaaatttgttgtaagcttaaatattgaattgttttcattttaagatagaatatcataatgtagactatctacagcttcattgtagattatacagatatatgatttctaaccttattactggaatttttcttccacagtaaaaatatatttgcattcttaatgctaattatctgcaagtattttttcattgtgtagtatgttgcagttactcatattaaacataacttgtatatttattattttaatgaagtttgagaataacgttacatatgttgaattttaagtactacagatttaactgattttatatttctgaaaggctaacagacatggatacacgtgtacagtatgcattcaaacttatttaaattggtgtatttttttttaagtcactgtccatttgtattgacatgcctctgtttctagtccagtttggagattttataaagttataacaatgagttaatgtgttcattttcatttgttgcatgtgacttaaatacagctagtatttggcattgagattttaatagaggttataattaacagttcctcttatagataataatctgggatccatgggtgggcttcagagaatctgtgtaccccctaaaattgtatgtagaattttggatatttacatttttattttttaactcttggatccatcagtaatatgtatttttctatatggtacaagatttaaggatctaattttatttttattccaaacagatagctatttgtctcaataccatttattaaacattctttctcccactg

SEQ ID NO: 2 - protein, 537 aa, Homo sapiens
MLARETYEEDREYESQAKRLKTEEGEIDYSAEEGENRREATPRGGGDGGGGGRSFSQPEAGGSHHKVSVSPVVHVRGLCESVVEADLVEALEKFGTICYVMMMPFKRQALVEFENIDSAKECVTFAADEPVYIAGQQAFFNYSTSKRITRPGNTDDPSGGNKVLLLSIQNPLYPITVDVLYTVCNPVGKVQRIVIFKRNGIQAMVEFESVLCAQKAKAALNGADIYAGCCTLKIEYARPTRLNVIRNDNDSWDYTKPYLGRRDRGKGRQRQAILGEHPSSFRHDGYGSHGPLLPLPSRYRMGSRDTPELVAYPLPQASSSYMHGGNPSGSVVMVSGLHQLKMNCSRVFNLFCLYGNIEKVKFMKTIPGTALVEMGDEYAVERAVTHLNNVKLFGKRLNVCVSKQHSVVPSQIFELEDGTSSYKDFAMSKNNRFTSAGQASKNIIQPPSCVLHYYNVPLCVTEETFTKLCNDHEVLTFIKYKVFDAKPSAKTLSGLLEWECKTDAVEALTALNHYQIRVPNGSNPYTLKLCFSTSSHL

SEQ ID NO: 3 - DNA, 1931 bp, Homo sapiens
  misc_feature (1917)..(1917): n is a, c, g, or t
ggcacgagggtaattgagaggagcggccgctccagagcttcctcccggggcgcccccctcagtccgtccgcgcttctcagccgccagtctcctggccgcgcagtccccgcggacggccgggccgcggagaccctcgcagaagaggcgctgccggcggcgaggactggccggcggcatccgaggcgcgcgccccaccgccacccccacccgcctgccagccggtccctccggccgccgccatgtcctcctcctcttcctcccccagggagacgtacgaggaggaccgggagtacgagagccaggccaagcgtctcaagaccgaggagggggagatcgactactcggccgaggaaggcgagaaccgccgggaagcgacgccccggggcgggggcgatggcggcggcggcggccggagcttctctcagccggaggcaggtggaagtcatcataaagtttctgtttcacccgtcgtccatgttcgaggactctgtgaatctgtggtggaagcagacctcgtggaagcgctggaaaaatttgggacaatatgctatgtgatgatgatgccatttaaacgacaggctctagtggaatttgaaaacatagatagtgccaaagaatgtgtgacatttgctgcagatgaacccgtgtacattgctggtcaacaggcttttttcaactattctacaagcaaaaggatcactcggccaggaaatactgatgatccatcaggaggcaacaaagttcttctgctctcaattcagaatccgctttatccaattacagtggatgttttatatactgtatgcaaccctgttggcaaagtgcaacgtattgttatattcaagagaaatgggatacaagcaatggttgatcgtctaaatgttattaggaatgacaatgacagttgggactacactaaaccatatttgggaagacgagatagaggaaagggtcgccagagacaagccattttgggagaacacccttcttcgtttagacatgatggctatggatcccatggtccattattgcctttaccaagtcgttacagaatgggctctcgagatacacctgaacttgttgcttatccattaccacaggcttcttcctcttacatgcatggaggaaatccctctggttcagttgtaatggttagtggattacatcaactaaaaatgaattgttcaagagtcttcaacctgttctgcttatatggaaatattgagaaggtaaaatttatgaagaccattcctggtacagcactggtagaaatgggtgatgagtatgctgtagaaagagctgtcacacaccttaataatgtcaaattatttgggaaaagacttaatgtttgcgtgtctaaacaacattcagttgttccaagtcaaatatttgagctggaggatggtaccagcagctacaaagattttgcaatgagcaaaaataatcgctttacaagtgctggccaagcatctaagaatataatccagccaccctcctgtgttttgcattattataatgttccattgtgtgtcacagaagagaccttcacaaagttgtgtaatgaccatgaagttcttacattcatcaaatataaagtgtttgatgcaaaaccttcagccaaaacactttctgggctattagaatgggagtgcaaaactgatgcagtagaagcccttacggcactgaatcactatcagataagagtgccgaatggttccaatccctatacattgaagctttgcttttctacatcatcccatttataagaagagaagagcatgttagaatttatgttcacctttattacaatttcaaagctacacttcattaaaaaaaaatctaaaatggttgatctcatgttgccttgcttactttaagatcctgttctgtaataaacatattttgccttgagtaaanaaanaaaaaaaaaa

SEQ ID NO: 4 - protein, 508 aa, Homo sapiens
MSSSSSSPRETYEEDREYESQAKRLKTEEGEIDYSAEEGENRREATPRGGGDGGGGGRSFSQPEAGGSHHKVSVSPVVHVRGLCESVVEADLVEALEKFGTICYVMMMPFKRQALVEFENIDSAKECVTFAADEPVYIAGQQAFFNYSTSKRITRPGNTDDPSGGNKVLLLSIQNPLYPITVDVLYTVCNPVGKVQRIVIFKRNGIQAMVDRLNVIRNDNDSWDYTKPYLGRRDRGKGRQRQAILGEHPSSFRHDGYGSHGPLLPLPSRYRMGSRDTPELVAYPLPQASSSYMHGGNPSGSVVMVSGLHQLKMNCSRVFNLFCLYGNIEKVKFMKTIPGTALVEMGDEYAVERAVTHLNNVKLFGKRLNVCVSKQHSVVPSQIFELEDGTSSYKDFAMSKNNRFTSAGQASKNIIQPPSCVLHYYNVPLCVTEETFTKLCNDHEVLTFIKYKVFDAKPSAKTLSGLLEWECKTDAVEALTALNHYQIRVPNGSNPYTLKLCFSTSSHL

SEQ ID NO: 5 - DNA, 20 bp, Artificial sequence
  Single strand DNA oligonucleotide
accacagtccatgccatcac

SEQ ID NO: 6 - DNA, 20 bp, Artificial sequence
  Single strand DNA oligonucleotide
tccaccaccctgttgctgta

SEQ ID NO: 7 - DNA, 17 bp, Artificial sequence
  Single strand DNA oligonucleotide
acaaagttcttctgctc

SEQ ID NO: 8 - DNA, 18 bp, Artificial sequence
  Single strand DNA oligonucleotide
attaaggtgtgtgacagc

SEQ ID NO: 9 - DNA, 20 bp, Artificial sequence
  Single strand DNA oligonucleotide
cagattctctgaagcccacc

SEQ ID NO: 10 - DNA, 21 bp, Artificial sequence
  Single strand DNA oligonucleotide
cattcctggtacagcactggt

SEQ ID NO: 11 - DNA, 2378 bp, Homo sapiens
ggttgcgtccccatccttagattcgctcctccgggcagggagcggagacggaggaggaggagggagaggctgaatgttggctcgggagacgtacgaggaggaccgggagtacgagagccaggccaagcgtctcaagaccgaggagggggagatcgactactcggccgaggaaggcgagaaccgccgggaagcgacgccccggggcgggggcgatggcggcggcggcggccggagcttctctcagccggaggcaggtggaagtcatcataaagtttctgtttcacccgtcgtccatgttcgaggactctgtgaatctgtggtggaagcagacctcgtggaagcgctggaaaaatttgggacaatatgctatgtgatgatgatgccatttaaacgacaggctctagtggaatttgaaaacatagatagtgccaaagaatgtgtgacatttgctgcagatgaacccgtgtacattgctggtcaacaggcttttttcaactattctacaagcaaaaggatcactcggccaggaaatactgatgatccatcaggaggcaacaaagttcttctgctctcaattcagaatccgctttatccaattacagtggatgttttatatactgtatgcaaccctgttggcaaagtgcaacgtattgttatattcaagagaaatgggatacaagcaatggttgagtttgaatcagtcctttgtgcccagaaagctaaagcagcactcaatggagctgatatatatgctggatgttgcacactaaaaattgaatatgcacggccaactcgtctaaatgttattaggaatgacaatgacagttgggactacactaaaccatatttgggaagacgagatagaggaaagggtcgccagagacaagccattttgggagaacacccttcttcgtttagacatgatggctatggatcccatggtccattattgcctttaccaagtcgttacagaatgggctctcgagatacacctgaacttgttgcttatccattaccacaggcttcttcctcttacatgcatggaggaaatccctctagttcagttgtaatggttagtggattacatcaactaaaaatgaattgttcaagagtcttcaacctgttctgcttatatggaaatattgagaaggtaaaatttatgaagaccattcctggtacagcactggtagaaatgggtgatgagtatgctgtagaaagagctgtcacacaccttaataatgtcaaattatttgggaaaagacttaatgtttgcgtgtctaaacaacattcagttgttccaagtcaaatatttgagctggaggatggtaccagcagctacaaagattttgcaatgagcaaaaataatcgctttacaagtgctggccaagcatctaagaatataatccagccaccctcctgtgttttgcattattataatgttccattgtgtgtcacagaagagaccttcacaaagttgtgtaatgaccatgaagttcttacattcatcaaatataaagtgtttgatgcaaaaccttcagccaaaacactttctgggctattagaatgggagtgcaaaactgatgcagtagaagcccttacggcactgaatcactatcagataagagtgccgaatggttccaatccctatacattgaagctttgcttttctacatcatcccatttataagaagagaagagcatgttagaatttatgttcacctttattacaatttcaaagctacacttcattaaaaaaaaatctaaaatggttgatctcatgttgccttgcttactttaagatcctgttctgtaataaacatattttgccttgagtaaatttgttgtaagcttaaatattgaattgttttcattttaagatagaatatcataatgtagactatctacagcttcattgtagattatacagatatatgatttctaaccttattactggaatttttcttccacagtaaaaatatatttgcattcttaatgctaattatctgcaagtattttttcattgtgtaagagattaatgcaggtgaaagtattgcattttaatatagaattcctattatatgtttagatgtttaagtatgttgcagttactcatattaaacataacttgtatatttattattttaatgaagtttgagaataacgttacatatgttgaattttaagtactacagatttaactgattttatatttctgaaaggctaacagacatggatacacgtgtacagtatgcattcaaacttatttaaattggtgtatttttttttaagtcactgtccatttgtattgacatgcctctgtttctagtccagtttggagattttataaagttataacaatgaaaaaaaaaaaaaaaaaa

SEQ ID NO: 12 - DNA, 2213 bp, Homo sapiens
gtggaagtcatcataaagtttctgtttcacccgtcgtccatgttcgaggactctgtgaatctgtggtggaagcagacctcgtggaagcgctggaaaaatttgggacaatatgctatgtgatgatgatgccatttaaacgacaggctctagtggaatttgaaaacatagatagtgccaaagaatgtgtgacatttgctgcagatgaacccgtgtacattgctggtcaacaggcttttttcaactattctacaagcaaaaggatcactcggccaggaaatactgatgatccatcaggaggcaacaaagttcttctgctctcaattcagaatccgctttatccaattacagtggatgttttatatactgtatgcaaccctgttggcaaagtgcaacgtattgttatattcaagagaaatgggatacaagcaatggttgagtttgaatcagtcctttgtgcccagaaagctaaagcagcactcaatggagctgatatatatgctggatgttgcacactaaaaattgaatatgcacggccaactcgtctaaatgttattaggaatgacaatgacagttgggactacactaaaccatatttgggaagacgaggttgttgttggcttatgctgaaacttcttgaaagaagcccactttgaaaatgtgttgctgttgaagctaatagtatgattcagatagaggaaagggtcgccagagacaagccattttgggagaacacccttcttcgtttagacatgatggctatggatcccatggtccattattgcctttaccaagtcgttacagaatgggctctcgagatacacctgaacttgttgcttatccattaccacaggcttcttcctcttacatgcatggaggaaatccctctggttcagttgtaatggttagtggattacatcaactaaaaatgaattgttcaagagtcttcaacctgttctgcttatatggaaatattgagaaggtaaaatttatgaagaccattcctggtacagcactggtagaaatgggtgatgagtatgctgtagaaagagctgtcacacaccttaataatgtcaaattatttgggaaaagacttaatgtttgcgtgtctaaacaacattcagttgttccaagtcaaatatttgagctggaggatggtaccagcagctacaaagattttgcaatgagcaaaaataatcgctttacaagtgctggccaagcatctaagaatataatccagccaccctcctgtgttttgcattattataatgttccattgtgtgtcacagaagagaccttcacaaagttgtgtaatgaccatgaagttcttacattcatcaaatataaagtgtttgatgcaaaaccttcagccaaaacactttctgggctattagaatgggagtgcaaaactgatgcagtagaagcccttacggcactgaatcactatcagataagagtgccgaatggttccaatccctatacattgaagctttgcttttctacatcatcccatttataagaagagaagagcatgttagaatttatgttcacctttattacaatttcaaagctacacttcattaaaaaaaaatctaaaatggttgatctcatgttgccttgcttactttaagatcctgttctgtaataaacatattttgccttgagtaaatttgttgtaagcttaaatattgaattgttttcattttaagatagaatatcataatgtagactatctacagcttcattgtagattatacagatatatgatttctaaccttattactggaatttttcttccacagtaaaaatatatttgcattcttaatgctaattatctgcaagtattttttcattgtgtaagagattaatgcaggtgaaagtattgcattttaatatagaattcctattatatgtttagatgtttaagtatgttgcagttactcatattaaacataacttgtatatttattattttaatgaagtttgagaataacgttacatatgttgaattttaagtactacagatttaactgattttatatttctgaaaggctaacagacatggatacacgtgtacagtatgcattcaaacttatttaaattggtgtatttttttttaagtcactgtccatttgtattgacatgcctctgtttctagtccagtttggagattttataaagttataacaatgaaaaaaaaaaaaaaaaaaaaaaaa

SEQ ID NO: 13 - DNA, 2055 bp, Homo sapiens
ggagggagaggctgaatgttggctcggtaattgagaggagcggccgctccagagcttcctcccggggcgcccccctcagtccgtccgcgcttctcagccgccagtctcctggccgcgcagtccccgcggacggccgggccgcggagaccctcgcagaagaggcgctgccggcggcgaggactggccggcggcatccgaggcgcgcgccccaccgccacccccacccgcctgccagccggtccctccggccgccgccatgtcctcctcctcttcctcccccagggagacgtacgaggaggaccgggagtacgagagccaggccaagcgtctcaagaccgaggagggggagatcgactactcggccgaggaaggcgagaaccgccgggaagcgacgccccggggcgggggcgatggcggcggcggcggccggagcttctctcagccggaggcaggtggaagtcatcataaagtttctgtttcacccgtcgtccatgttcgaggactctgtgaatctgtggtggaagcagacctcgtggaagcgctggaaaaatttgggacaatatgctatgtgatgatgatgccatttaaacgacaggctctagtggaatttgaaaacatagatagtgccaaagaatgtgtgacatttgctgcagatgaacccgtgtacattgctggtcaacaggcttttttcaactattctacaagcaaaaggatcactcggccaggaaatactgatgatccatcaggaggcaacaaagttcttctgctctcaattcagaatccgctttatccaattacagtggatgttttatatactgtatgcaaccctgttggcaaagtgcaacgtattgttatattcaagagaaatgggatacaagcaatggttgagtttgaatcagtcctttgtgcccagaaagctaaagcagcactcaatggagctgatatatatgctggatgttgcacactaaaaattgaatatgcacggccaactcgtctaaatgttattaggaatgacaatgacagttgggactacactaaaccatatttgggaagacgagatagaggaaagggtcgccagagacaagccattttgggagaacacccttcttcgtttagacatgatggctatggatcccatggtccattattgcctttaccaagtcgttacagaatgggctctcgagatacacctgaacttgttgcttatccattaccacaggcttcttcctcttacatgcatggaggaaatccctctggttcagttgtaatggttagtggattacatcaactaaaaatgaattgttcaagagtcttcaacctgttctgcttatatggaaatattgagaaggtaaaatttatgaagaccattcctggtacagcactggtagaaatgggtgatgagtatgctgtagaaagagctgtcacacaccttaataatgtcaaattatttgggaaaagacttaatgtttgcgtgtctaaacaacattcagttgttccaagtcaaatatttgagctggaggatggtaccagcagctacaaagattttgcaatgagcaaaaataatcgctttacaagtgctggccaagcatctaagaatataatccagccaccctcctgtgttttgcattattataatgttccattgtgtgtcacagaagagaccttcacaaagttgtgtaatgaccatgaagttcttacattcatcaaatataaagtgtttgatgcaaaaccttcagccaaaacactttctgggctattagaatgggagtgcaaaactgatgcagtagaagcccttacggcactgaatcactatcagataagagtgccgaatggttccaatccctatacattgaagctttgcttttctacatcatcccatttataagaagagaagagcatgttagaatttatgttcacctttattacaatttcaaagctacacttcattaaaaaaaaatctaaaatggttgatctcatgttgccttgcttactttaagatcctgttctgtaataaacatattttgccttgagtaaaaaaaaaaaaaaaaaaaaaaa

SEQ ID NO: 14 - DNA, 66 bp, Homo sapiens
  misc_feature: EXON 7 of the HNRPLL gene
agagattaatgcaggtgaaagtattgcattttaatatagaattcctattatatgtttagatgttta

SEQ ID NO: 15 - DNA, 512 bp, Homo sapiens
  misc_feature: EXON 1 the HNRPLL gene (excluded from transcript A)
ggttgcgtccccatccttagattcgctcctccgggcagggagcggagacggaggaggaggagggagaggctgaatgttggctcggtaattgagaggagcggccgctccagagcttcctcccggggcgcccccctcagtccgtccgcgcttctcagccgccagtcagccgccagcgcagtccccgcggacggccgggccgcggagaccctcgcagaagaggcgctgccggcggcgaggactggccggcggcatccgaggcgcgcgccccaccgccacccccacccgcctgccagccggtccctccggccgccgccatgtcctcctcctcttcctcccccagggagacgtacgaggaggaccgggagtacgagagccaggccaagcgtctcaagaccgaggagggggagatcgactactcggccgaggaaggcgagaaccgccgggaagcgacgccccggggcgggggcgatggcggcggcggcggccggagcttctctcagccggtaacaaag

SEQ ID NO: 16 - DNA, 102 bp, Homo sapiens
  misc_feature: Exon 5 of the HNRPLL gene (excluded from transcript B)
gtttgaatcagtcctttgtgcccagaaagctaaagcagcactcaatggagctgatatatatgctggatgttgcacactaaaaattgaatatgcacggccaac

SEQ ID NO: 17 - DNA, 167 bp, Homo sapiens
  misc_feature: EXON 14 of transcript A (HNRPLL gene)
tgtatgtagaattttggatatttacatttttattttttaactcttggatccatcagtaatatgtatttttctatatggtacaagatttaaggatctaattttatttttattccaaacagatagctatttgtctcaataccatttattaaacattctttctcccactg

SEQ ID NO: 18 - DNA, 8 bp, Homo sapiens
  misc_feature: HNRPLL gene transcript B unique sequence (exon 1 partial sequence)
ggcacgag